(19) United States
(12) Patent Application Publication
(10) Pub. No.: US 2026/0232455 A1
(43) Pub. Date: Aug. 13, 2026

(54) HUMERAL RECONSTRUCTION PROSTHESIS

(71) Applicant: FX SHOULDER SOLUTIONS, Viriat (FR)

(72) Inventors: Raúl BARCO, Madrid (ES); Julien DERANLOT, Paris (FR); Robert FULLICK, Houston, TX (US); Herrick SIEGEL, Mountain Brook, AL (US); Umasuthan SRIKUMARAN, Ellicott City, MD (US)

(73) Assignee: FX SHOULDER SOLUTIONS, Viriat (FR)

(21) Appl. No.: 19/537,724

(22) Filed: Feb. 12, 2026

(30) Foreign Application Priority Data
Feb. 12, 2025 (FR) FR2501462

(51) Int. Cl. A61F 2/40; A61F 2/28; A61F 2/30

(52) U.S. Cl. CPC A61F 2/4059; A61F 2002/2853; A61F 2002/30433; A61F 2002/30616; A61F 2002/4062; A61F 2002/4074; A61F 2002/4077

(57) ABSTRACT

A humeral prosthesis including: a distal part including a device for attaching to a resected humerus, a metaphyseal part including a device for attaching a joint element of a gleno-humeral joint, a diaphyseal part configured to replace a part of resected humerus, extending between the distal part and the metaphyseal part and being attached to the distal part and to the metaphyseal part.

### TECHNICAL FIELD

The invention relates to humeral reconstruction prostheses, in particular following significant bone loss.

### STATE OF THE ART

Some patients may suffer from more or less significant humeral bone loss, for example due to the appearance of a tumor in the proximal part of the humerus requiring bone resection.

In this case, the distal part of the resected humerus must be replaced to reconstruct the humerus and recreate a joint with the glenoid.

The invention aims to provide a humeral prosthesis allowing partial reconstruction of the humerus with a certain degree of flexibility during the reconstruction of the humerus, a reconstruction that is stable and allows a patient to recover a functional gleno-humeral joint while eliminating any pain.

### SUMMARY OF THE INVENTION

This invention therefore relates to a humeral prosthesis comprising:

a distal part comprising means for attaching to a resected humerus,

a metaphyseal part comprising means for attaching a joint element of a gleno-humeral joint, and

a diaphyseal part configured to replace a part of resected humerus, extending between the distal part and the metaphyseal part and being attached to the distal part and to the metaphyseal part.

We thus obtain a prosthesis allowing humeral reconstruction regardless of the humeral length to be reconstructed. In fact, the presence of a diaphyseal part independent of the distal and metaphyseal parts makes it possible to adapt the diaphyseal part to the patient requiring humeral reconstruction.

The distal part is used to attach the entire humeral prosthesis to a resected humerus, in a stable manner. Being independent with respect to the rest of the humeral prosthesis, a distal part adapted to the case to be dealt with by the surgeon can be chosen.

Lastly, the metaphyseal part, that is also independent of the other parts of the humeral prosthesis, can be chosen depending on the patient.

According to other optional characteristics of the humeral prosthesis taken alone or in combination:

the distal part comprises a rod comprising means for attaching to the diaphyseal part and intended to extend inside a medullary cavity of a resected humerus,

the distal part comprises a flange placed on the rod and intended to surround at least partially one end of a resected humerus,

the flange and the rod comprise first means for indexing the flange on the rod,

the flange and the rod comprise holes for locking the distal part to a humerus, the locking holes being intended to be aligned and crossed by a locking screw when mounting the distal part on a resected humerus,

the hole locking the rod is oblong,

the one or more holes locking the flange comprise a thread interrupted by notches,

the rod comprises anchoring holes intended to be invaded by a bone material of the resected humerus,

the diaphyseal part comprises stitching holes,

the diaphyseal part comprises a first mounting hole on the distal part cooperating with a first mounting stud carried by the distal part,

the mounting hole and the mounting stud comprise second means for indexing the diaphyseal part on the distal part,

the metaphyseal part comprises a second mounting hole on the diaphyseal part cooperating with a second mounting stud carried by the diaphyseal part,

the metaphyseal part can be rotated on the diaphyseal part when mounting the metaphyseal part on the diaphyseal part,

the metaphyseal part comprises a flat proximal end configured to receive a joint element of a gleno-humeral joint,

the flat proximal end extends in a plane inclined relative to an extension axis of the diaphyseal part at an angle of between 30° and 60°, preferably between 40° and 50°, preferably substantially equal to 45°,

A locking screw extends from the metaphyseal part up to the distal part and locks the distal, diaphyseal and metaphyseal parts together,

The locking screw comprises a threaded end screwed into a threaded hole formed in the distal part, and

The locking screw comprises a stop pressing against a shoulder of the metaphyseal part.

The invention also relates to an implantation kit for the design of a humeral prosthesis according to the invention, the kit comprising:

Several distal parts of different length and/or width,

Several metaphyseal parts of different length and/or width, and

Several diaphyseal parts of different length and/or width.

According to other optional characteristics of the implantation kit taken alone or in combination:

the number and/or position of the locking holes in the flange and/or the rod differ from one distal part to another,

the length of the flange differs from one distal part to another,

the length of the rod differs from one distal part to another,

the number and/or position of the anchoring holes in the rod differ from one distal part to another,

the number and/or position of the stitching holes in the diaphyseal part differ from one diaphyseal part to another, and

the flat proximal end extends in a plane whose inclination relative to an extension axis of the diaphyseal part differs from one metaphyseal part to another.

### BRIEF DESCRIPTION OF THE FIGURES

The invention will be better understood on reading the following description, given solely by way of example and with reference to the accompanying drawings in which:

FIG. 1 is a perspective view of a humeral prosthesis according to a first embodiment of the invention,

FIG. 2 is an exploded view of a humeral prosthesis according to the first embodiment of the invention,

FIG. 3 is a longitudinal cross-sectional view of a humeral prosthesis according to the first embodiment of the invention,

FIG. 4 is a perspective view of a distal part according to the first embodiment of the invention,

FIG. 5 is a longitudinal cross-sectional view of a distal part according to the first embodiment of the invention,

FIG. 6 is a perspective view of the upper portion of a rod of a distal part according to the first embodiment of the invention,

FIG. 7 is a perspective view of a flange of a distal part according to the first embodiment of the invention,

FIG. 8 is a perspective view of a lower portion of a flange of a distal part according to the first embodiment of the invention,

FIG. 9 is a perspective view of a portion of a distal part according to the first embodiment of the invention,

FIG. 10 is a perspective view of a humeral prosthesis according to a second embodiment of the invention, and

FIG. 11 is an exploded view of a humeral prosthesis according to the second embodiment of the invention.

### DETAILED DESCRIPTION

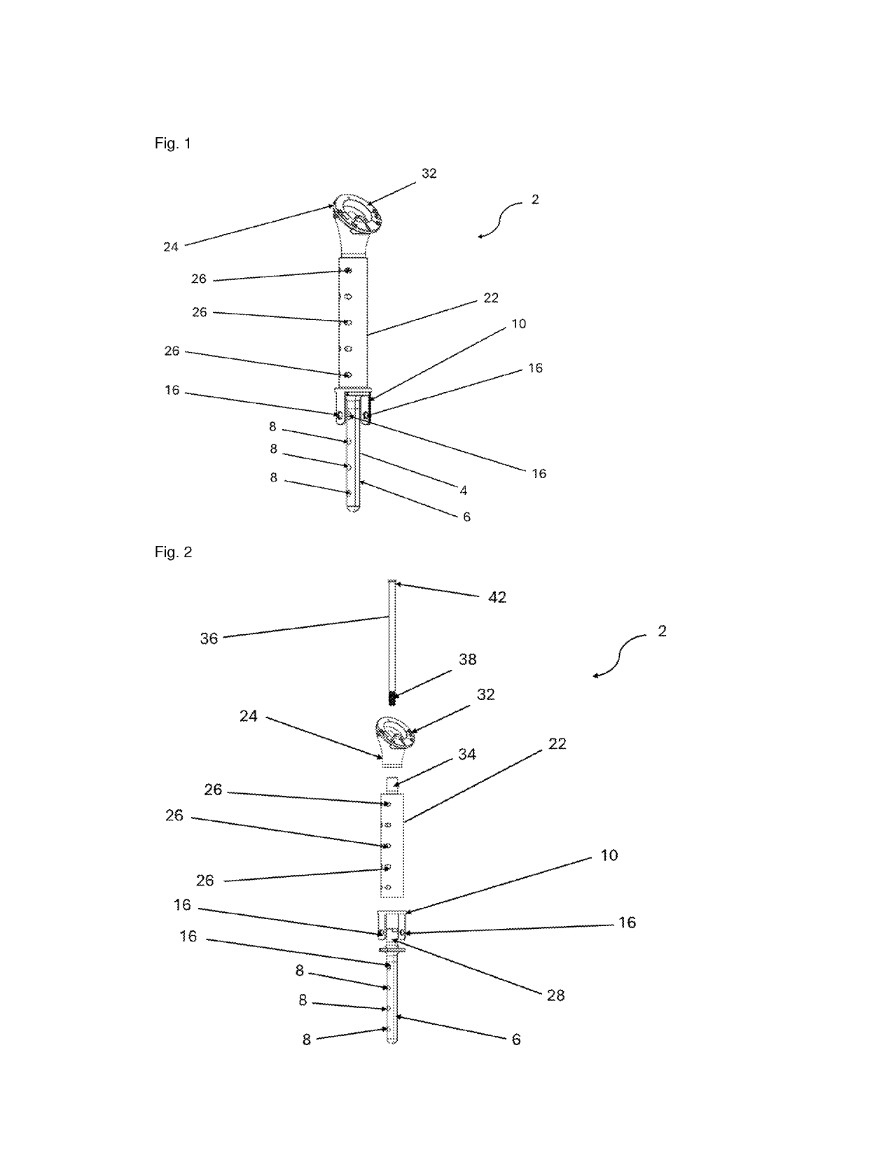

We now refer to FIG. 1 showing a first embodiment of a humeral prosthesis 2. As explained above, this prosthesis is used to reconstruct a humerus of a patient who has undergone more or less significant resection of the humerus, in particular following a cancer or tumor.

The humeral prosthesis 2 is typically made of a biocompatible material, for example a chromium-cobalt-molybdenum alloy, an austenitic steel or a titanium alloy.

The humeral prosthesis 2 comprises firstly a distal part 4 comprising means for attaching to a resected humerus. This part is intended to create a connection with a resected humerus and therefore form an anchoring base for the other parts of the humeral prosthesis 2.

The distal part 4 can consist of a rod 6, comprising means for attaching to a diaphyseal part 22 (described below) and intended to extend inside a medullary cavity of a resected humerus. As explained below, the attachment means can consist of a first mounting stud 28.

The rod 6 can therefore be used to anchor the humeral prosthesis inside the resected humerus. This is possible since it extends over a sufficient length in the resected humerus. In addition to this extension, the rod 6 can comprise additional elements used to anchor the distal part 4 in the resected humerus. As shown on the figures, the rod 6 can comprise anchoring holes 8 intended to be crossed by attachment screws to anchor the rod 6 securely in the bone. As an alternative, the rod 6 could be coated by a material and/or comprise shapes on its surface in order to allow anchoring by cooperation with the patient's bone.

The distal part 4 can comprise a flange 10 placed on the rod 6 and intended to surround at least partially one end of a resected humerus. In other words, the flange 10 extends around the bone whereas the rod 6 extends inside the bone in order to stabilize the distal part 4, and therefore the humeral prosthesis 2, in the resected humerus.

For stabilization purposes, a flange 10 can be chosen with dimensions allowing surface contact between itself and the outer surface of the resected humerus.

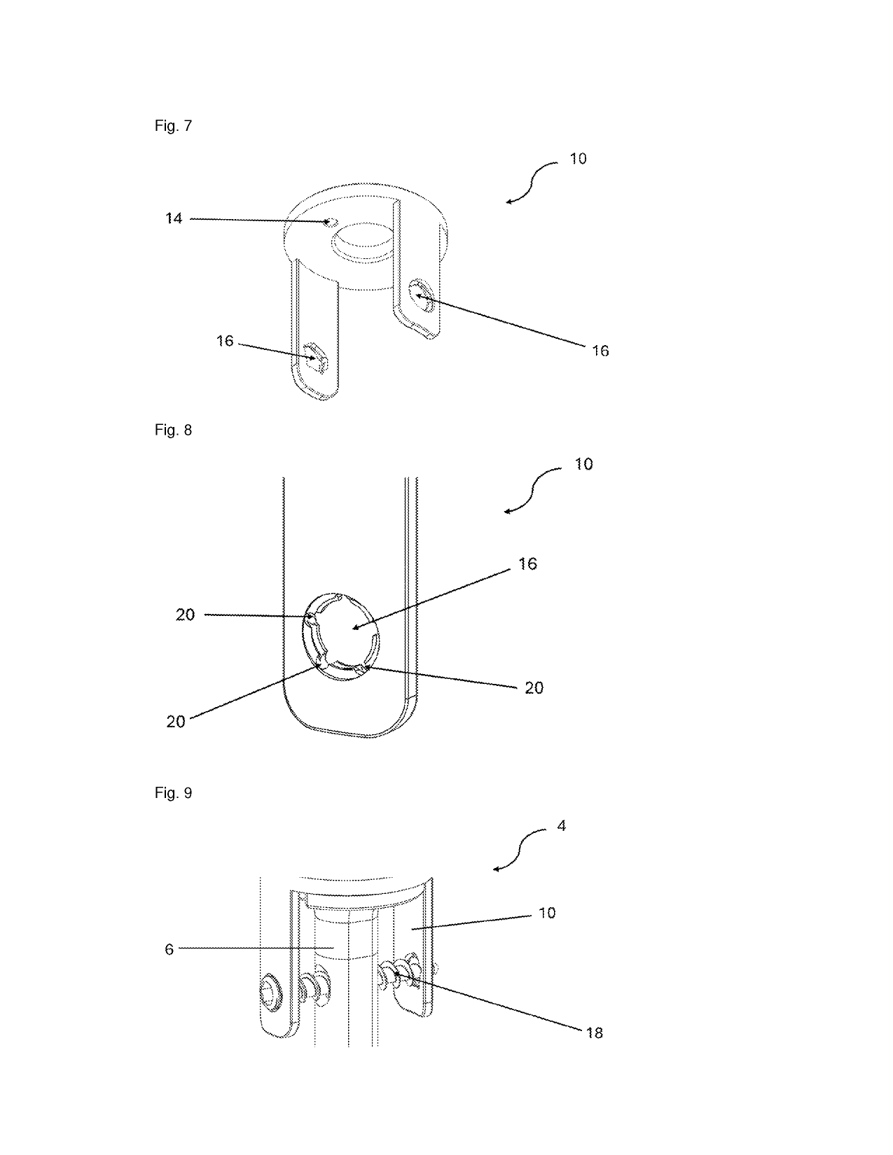

The flange 10 and the rod 6 can comprise first means for indexing the flange 10 on the rod 6. As shown on FIGS. 6 and 7, the rod can comprise a shoulder 11 having Morse cones 12 inserted in first indexing holes 14 when the flange 10 is placed on the rod 6. Obviously, the shape and/or number of the indexing means can vary.

The flange 10 and the rod 6 can comprise holes 16 for locking the distal part 4 to a humerus, the locking holes 16 being intended to be aligned and crossed by a locking screw 18. In other words, the rod comprises at least one locking hole 16 present inside the medullary cavity and the flange 10 comprises, at its one or more extensions surrounding the end of the resected humerus, one or more locking holes 16. When implanting the distal part 4, these locking holes 16 are aligned in order to use a locking screw 18 (shown on FIG. 9 for example) which will cross the humerus from one side to the other through the locking holes 16 to create an additional point for locking the distal part 4 in the bone. The locking screw 18 can be a compression bone screw or a screw locked without compression.

Several pairs of locking holes 16 can be created at different heights of the rod 6 and of the flange 10.

Advantageously, the hole 16 locking the rod 6 is oblong. This provides a mounting tolerance while locking the rotation of the distal part 4. Translational locking can be provided by another cylindrical hole present in the diaphyseal part 22.

Advantageously, the one or more holes 16 locking the flange comprise a thread interrupted by notches 20. These notches 20 are shown on FIG. 8 (five notches on FIG. 8). These notches can be filled with biological tissues in order to lock the locking screw 18.

As explained in the above description, the positioning and locking of the distal part 4 on the resected humerus are independent of the rest of the humeral prosthesis 2.

The humeral prosthesis 2 further comprises a diaphyseal part 22. This diaphyseal part 22 forms the main body of the humeral prosthesis 2 largely replacing the diaphyseal part of the resected humerus. It extends between the distal part 4 and a metaphyseal part 24 described below and is attached to these parts.

The diameter and length of the diaphyseal part 22 are chosen according to the patient's bone, considering the diameter of the resected humerus and the length of the resected part to be reconstructed.

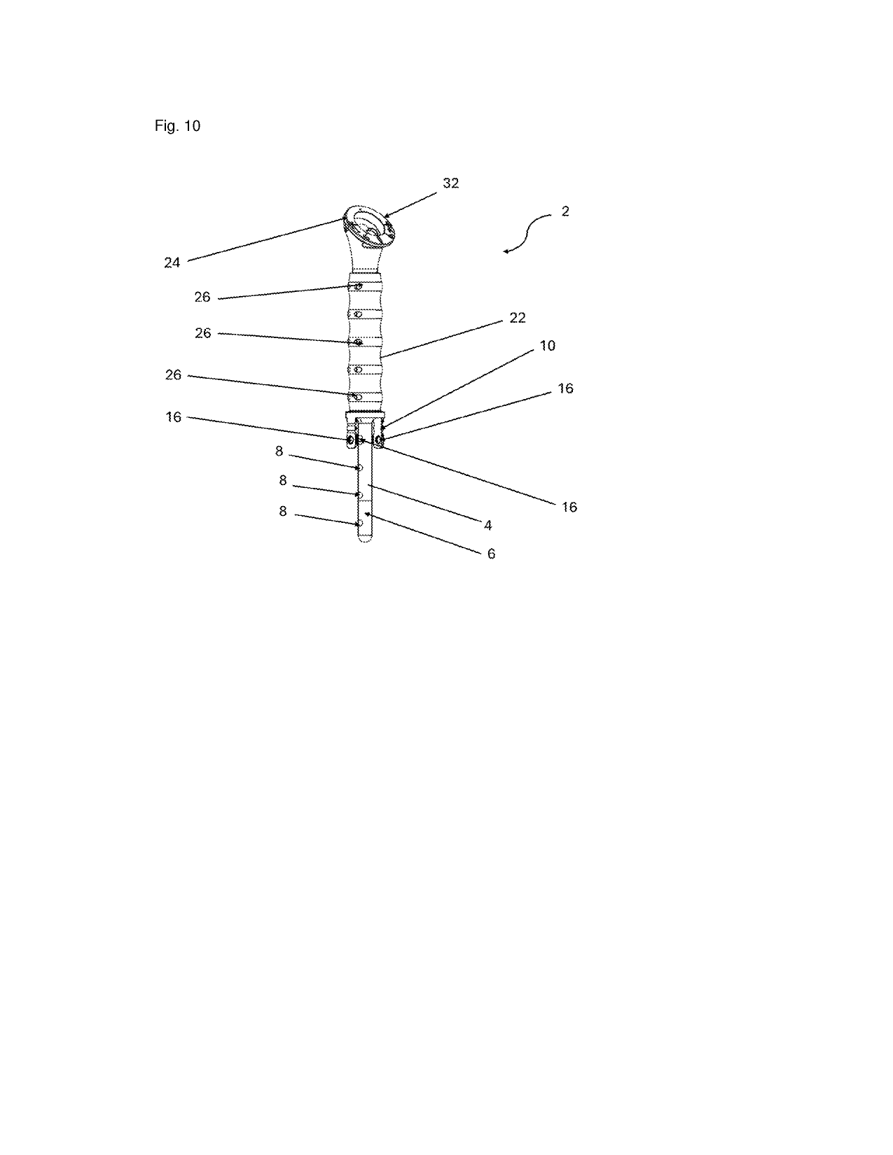

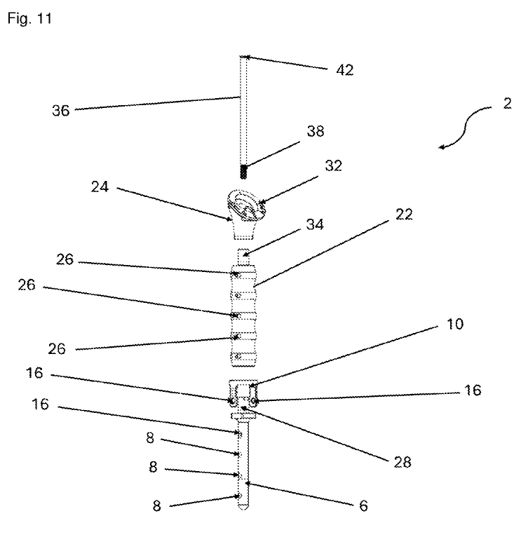

Advantageously, the diaphyseal part 22 can comprise holes 26 for stitching the deltoid muscle, and more generally the soft tissues, to the diaphyseal part 22. FIGS. 10 and 11 show a second embodiment of the humeral prosthesis according to the invention. In this embodiment, the outer sides of the diaphyseal part 22 and of the flange 10 are not smooth but have a variable width in order to position soft tissue stitching threads.

The diaphyseal part 22 can comprise a first mounting hole on the distal part 4 cooperating with a first mounting stud 28 (shown on FIGS. 2 to 6) carried by the distal part 4. Advantageously, the first mounting hole and the first mounting stud 28 comprise second means for indexing the diaphyseal part 22 on the distal part 4. FIG. 6 shows an indexing example with the presence of flats 30 on the first mounting stud 28 cooperating with shoulders present in the first mounting hole. We can also see that the flange 10 is mounted on the rod 6, being arranged around the first mounting stud 28, thanks to the presence of a through-hole formed in the flange 10.

The humeral prosthesis 2 also comprises a metaphyseal part 24 comprising means for attaching a joint element of a gleno-humeral joint. In other words, the metaphyseal part 24, which forms the most proximal part of the humeral prosthesis 2, forms a part for receiving a part of the joint to be formed with the glenoid. The joint element of a gleno-humeral joint is chosen from a cup (when installing a reverse joint prosthesis with installation of a glenosphere at the glenoid) or a humeral head (when installing an anatomical joint prosthesis with installation of a concave glenoid implant in the glenoid). The joint element of the gleno-humeral joint is chosen depending on the type of joint prosthesis that is itself chosen depending on the patient.

Advantageously, the metaphyseal part 24 comprises a flat proximal end 32 configured to receive a joint element of a gleno-humeral joint. The flat proximal end 32 can extend in a plane inclined relative to an extension axis of the diaphyseal part 22 at an angle of between 30° and 60°, preferably between 40° and 50°, preferably substantially equal to 45°.

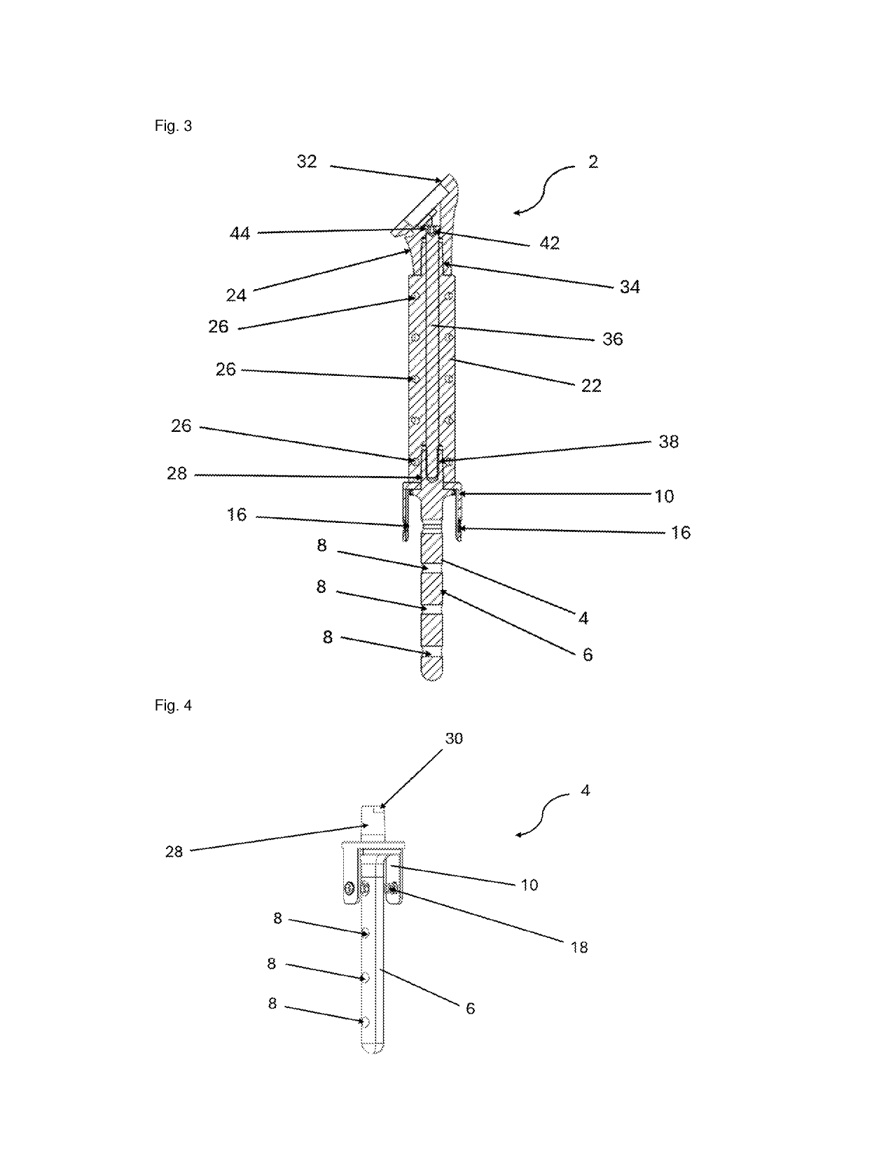

Advantageously, the metaphyseal part 24 comprises a second mounting hole on the diaphyseal part 22 cooperating with a second mounting stud 34 (shown on FIGS. 2 and 3) carried by the diaphyseal part 22.

Unlike what has been described regarding the relation between the distal part 4 and the diaphyseal part 22, the metaphyseal part 24 can be rotated on the diaphyseal part 22 when mounting the metaphyseal part 24 on the diaphyseal part 22. In other words, an indexing element is not necessarily present between these two parts so that the metaphyseal part 24 can be oriented according to the patient.

Advantageously, the humeral prosthesis 2 comprises a locking screw 36 extending from the metaphyseal part 24 up to the distal part 4 and locking the distal 4, diaphyseal 22 and metaphyseal 24 parts together. Thus, a single element crossing all the parts of the humeral prosthesis 2 can lock all the parts together.

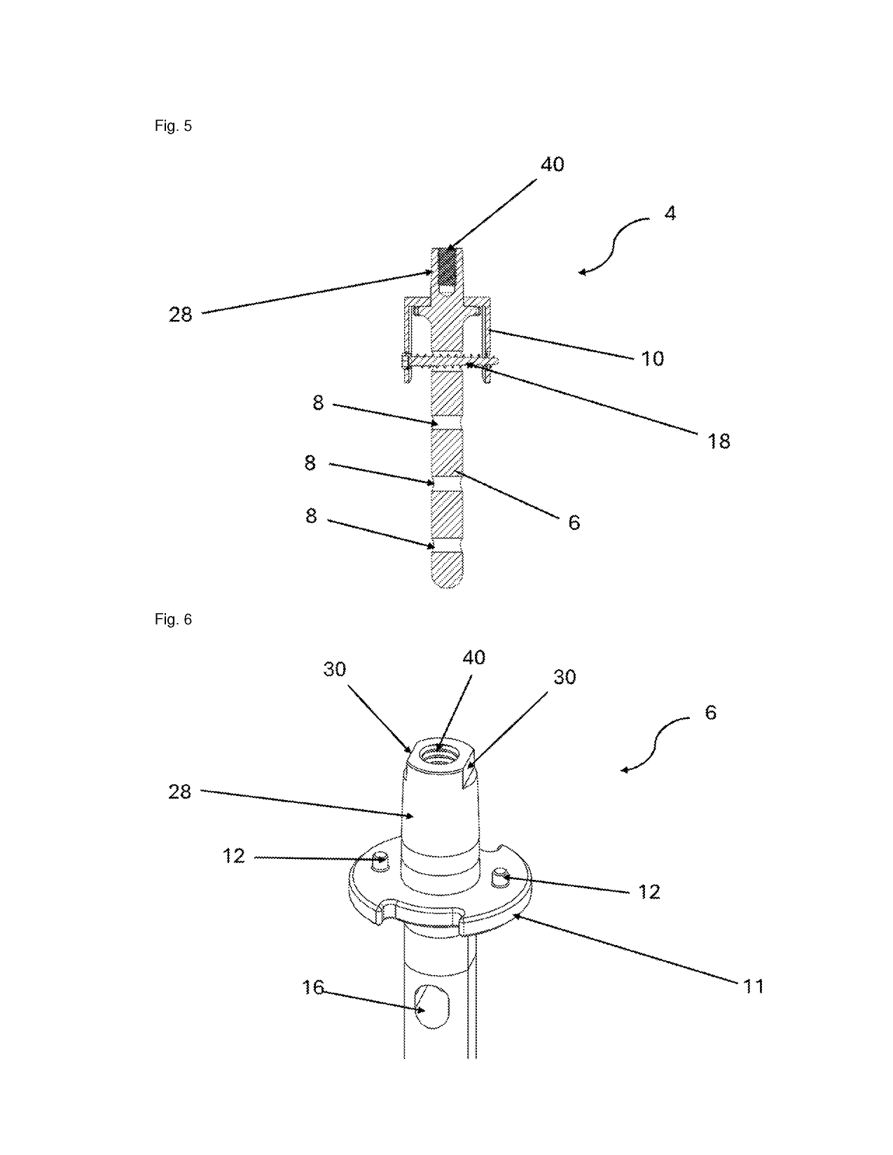

The locking screw 36 can comprise a threaded end 38 (shown on FIG. 2) screwed into a threaded hole 40 (shown on FIGS. 5 and 6) formed in the distal part 4. It can also comprise a stop 42 pressing against a shoulder 44 of the metaphyseal part 24. The attachment can now be carried out by screwing until the stop 42 comes up against the shoulder 44. If there is no stop 42 and no shoulder 44, the threaded hole 40 can be a blind hole where the threaded end 38 of the locking screw 36 comes up against the bottom.

The invention also relates to an implantation kit for the design of a humeral prosthesis 2 as described above, the kit comprising:

Several distal parts 4 of different length and/or width,

Several metaphyseal parts 24 of different length and/or width, and

Several diaphyseal parts 22 of different length and/or width.

Such a kit can therefore be used to choose parts having special dimensional properties to create a humeral prosthesis 2 adapted to each patient.

When selecting parts of a humeral prosthesis 2 that are adapted to a patient, it is possible that:

The number and/or position of the locking holes 16 in the flange 10 and/or the rod 6 differ from one distal part 4 to another.

The length of the flange 10 differs from one distal part 4 to another.

The length of the rod 6 differs from one distal part 4 to another.

The number and/or position of the anchoring holes 8 in the rod 6 differ from one distal part 4 to another.

The number and/or position of the stitching holes 26 in the diaphyseal part 22 differ from one diaphyseal part 22 to another.

The flat proximal end 32 extends in a plane whose inclination relative to an extension axis of the diaphyseal part 22 differs from one metaphyseal part 24 to another.

### List of references

2: humeral prosthesis

4: distal part

6: rod

8: anchoring holes

10: flange

11: first shoulder

12: Morse cones

14: first indexing holes

16: locking holes

18: locking screw

20: notches

22: diaphyseal part

24: metaphyseal part

26: stitching holes

28: first mounting stud

30: flats

32: flat proximal end

34: second mounting stud

36: locking screw

38: threaded end

40: threaded hole

42: stop

44: shoulder

What is claimed is:

1. A humeral prosthesis comprising:
a distal part comprising a device for attaching to a resected humerus,
a metaphyseal part comprising a device for attaching a joint element of a gleno-humeral joint,
a diaphyseal part configured to replace a part of resected humerus, extending between the distal part and the metaphyseal part and being attached to the distal part and to the metaphyseal part.

2. The humeral prosthesis according to claim 1, wherein the distal part comprises a rod comprising a device for attaching to the diaphyseal part and intended to extend inside a medullary cavity of a resected humerus.

3. The humeral prosthesis according to claim 2, wherein the distal part comprises a flange placed on the rod and intended to surround at least partially one end of a resected humerus.

4. The humeral prosthesis according to claim 3, wherein the flange and the rod comprise first device for indexing the flange on the rod.

5. The humeral prosthesis according to claim 3, wherein the flange and the rod comprise holes for locking the distal part to a humerus, the locking holes being intended to be aligned and crossed by a locking screw when mounting the distal part on a resected humerus.

6. The humeral prosthesis according to claim 1, wherein the diaphyseal part comprises a first mounting hole on the distal part cooperating with a first mounting stud carried by the distal part.

7. The humeral prosthesis according to claim 6, wherein the mounting hole and the mounting stud comprise second device for indexing the diaphyseal part on the distal part.

8. The humeral prosthesis according to claim 1, wherein the metaphyseal part comprises a second mounting hole on the diaphyseal part cooperating with a second mounting stud carried by the diaphyseal part.

9. The humeral prosthesis according to claim 1, wherein the metaphyseal part can be rotated on the diaphyseal part when mounting the metaphyseal part on the diaphyseal part.

10. The humeral prosthesis according to claim 1, wherein the metaphyseal part comprises a flat proximal end configured to receive a joint element of a gleno-humeral joint.

11. The humeral prosthesis according to claim 1, comprising a locking screw extending from the metaphyseal part up to the distal part and locking the distal, diaphyseal and metaphyseal parts together.

12. The humeral prosthesis according to claim 11, wherein the locking screw comprises a threaded end screwed into a threaded hole formed in the distal part.

13. The humeral prosthesis according to claim 11, wherein the locking screw comprises a stop pressing against a shoulder of the metaphyseal part.

14. An implantation kit for the design of a humeral prosthesis according to claim 1, the kit comprising:
several distal parts of different length and/or width,
several metaphyseal parts of different length and/or width, and
several diaphyseal parts of different length and/or width.
